(19) United States
(12) Patent Application Publication
(10) Pub. No.: US 2023/0374085 A1
(43) Pub. Date: Nov. 23, 2023

(54) DUAL-TARGETING CHIMERIC ANTIGEN RECEPTOR MODIFIED T CELLS COMPRISING IL-13 AND CHLOROTOXIN FOR CANCER TREATMENT

(71) Applicant: City of Hope, Duarte, CA (US)

(72) Inventors: Christine E. Brown, Duarte, CA (US); Dongrui Wang, Duarte, CA (US); Michael Barish, Duarte, CA (US); Stephen J. Forman, Duarte, CA (US)

(21) Appl. No.: 17/910,459

(22) PCT Filed: Mar. 11, 2021
(86) PCT No.: PCT/US2021/021890

§ 371 Date: Sep. 9, 2022

Related U.S. Application Data
(60) Provisional application No. 62/988,199, filed on Mar. 11, 2020.

(51) Int. Cl. C07K 14/435; C07K 14/54; A61K 39/00; C07K 14/725; A61P 35/00

(52) U.S. Cl. CPC C07K 14/43522; C07K 14/5437; A61K 39/4631; C07K 14/7051; A61K 39/4611; A61P 35/00; A61K 39/464419; A61K 39/464402; C07K 2319/33; C07K 2319/03; C07K 2319/02; A61K 2239/21; A61K 2239/22; A61K 2239/29

(57) ABSTRACT

Chimeric antigen receptors having a chlorotoxin domain and an IL-13 are described. These dual targeted chimeric antigen receptors are useful for treating glioblastoma and other cancers of neuroectodermal origin.

### CLAIM OF PRIORITY

This application is a U.S. National Phase Application under 35 U.S.C. § 371 of International Application No. PCT/US2021/021890, filed on Mar. 11, 2021, which claims the benefit of U.S. Provisional Application Ser. No. 62/988,199, filed on Mar. 11, 2020. The entire contents of the foregoing are incorporated herein by reference.

### SEQUENCE LISTING

This application contains a Sequence Listing that has been submitted electronically as an ASCII text file named 40056_0047US1_Sequence_Listing.txt and is hereby incorporated in reference in its entirety. Said ASCII text file, created on Sep. 8, 2022, is 79,718 bytes in size.

### TECHNICAL FIELD

This disclosure concerns bispecific chimeric antigen receptor (CAR)-engineered T cells comprising chlorotoxin (or variants thereof) and IL-13 (or variants thereof), methods of formulating, and methods of use as anti-cancer agents.

### BACKGROUND

IL-13Rα2 is highly expressed in several human tumors associated with poor prognosis, including primary brain tumors (e.g., gliomas (e.g., anaplastic astrocytoma (AA-grade III) and glioblastoma multiforme (GBM-grade IV)), pancreatic cancer, colorectal cancer, etc.) (Davis F G, McCarthy B J. Epidemiology of brain tumors. Curr Opin Neural. 2000; 13:635-640; Davis F G, Malinski N, Haenszel W, et al. Primary brain tumor incidence rates in four United States regions, 1985-1989: a pilot study. Neuroepidemiology. 1996; 15:103-112; Smith M A, Freidlin B, Ries L A, Simon R. Increased incidence rates but no space-time clustering of childhood astrocytoma in Sweden, 1973-1992: a population-based study of pediatric brain tumors. Cancer. 2000; 88:1492-1493; Davis F G, Freels S, Grutsch J, Barias S, Brem S. Survival rates in patients with primary malignant brain tumors stratified by patient age and tumor histological type: an analysis based on Surveillance, Epidemiology, and End Results (SEER) data, 1973-1991. J Neurosurg. 1998, 88:1-10; Fujisawa T, Joshi B, Nakajima A, et al (2009) A novel role of interleukin-13 receptor alpha2 in pancreatic cancer invasion and metastasis. Cancer Res 69: 8678-8685; Zhou R, et al. Interleukin-13 and its receptors in colorectal cancer, Biomedical Reports 1: 687-690, 2013).

Chlorotoxin possesses targeting properties towards cancer cells including glioma, melanoma, small cell lung carcinoma, neuroblastoma, and medulloblastoma (Dardevet L, et al. (2015) Chlorotoxin: A Helpful Natural Scorpion Peptide to Diagnose Glioma and Fight Tumor Invasion. Toxins, 7, 1079-1101). for IL-13 and a receptor for chlorotoxin. Furthermore, there is

[symbol]

The majority of CAR tumor-targeting domains are single chain variable fragments (scFvs) derived from antibody sequences that exploit the specificity of antibody binding to particular antigens. There are few examples of CAR tumor targeting domains derived from receptor ligands, and despite some notable successes, the identification and validation of novel CAR tumor targeting domains remains a major challenge in the field. There are currently no known CAR therapies featuring two tumor-targeting domains derived from receptor ligands.

### SUMMARY

Described herein are methods for using and making bispecific (also called dual-targeting) CAR T cells comprising both chlorotoxin (or a variant thereof) and IL-13 (or a variant thereof) to treat a variety of cancers, for example, gliomablastoma. These dual targeting CAR are generally referred to as CLTX/IL-13 CAR or IL-13/CLTX CAR throughout the disclosure.

Described herein is a nucleic acid molecule comprising a nucleotide sequence encoding a chimeric antigen receptor (CAR), wherein the chimeric antigen receptor comprises: a tumor-targeting domain comprising two subdomains (e.g., a chlorotoxin domain and an IL-13 domain in either order), a linker between the two tumor-targeting subdomains, a spacer, a transmembrane domain, a co-stimulatory domain, and a CD3ζ signaling domain.

In various embodiments: the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; the spacer comprises 20-150 amino acids and is located between the tumor-targeting domains and the transmembrane domain; the transmembrane domain is a CD4 transmembrane domain or variant thereof having 1-5 amino acid modifications; the transmembrane domain is a CD4 transmembrane domain; the chimeric antigen receptor comprises a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications; the spacer region comprises an amino acid sequence selected from the group consisting of SEQ ID NOs: 2-12 or a variant thereof having 1-5 amino acid modifications; the spacer comprises an IgG hinge region; the spacer comprises 10-50 amino acids; the CD28 costimulatory domains comprises the amino acid sequence of SEQ ID NO:22 or 23; the 4-1BB costimulatory domain comprises the amino acid sequence of SEQ ID NO: 24 or a variant thereof having 1-5 amino acid modifications; the CD3ζ signaling domain comprises the amino acid sequence of SEQ ID NO:21; a linker of 3 to 15 amino acids is located between the 4-1BB costimulatory domain and the CD3ζ signaling domain or the CD28 costimulatory domain and the CD3ζ signaling domain, or variant thereof; the CAR comprises the amino acid sequence of any one of SEQ ID NOS: 29-33 or a variant thereof having 1-5 amino acid modifications; the tumor targeting domain comprises the amino acid sequence of any one of SEQ ID NOS: 1, 34-42 or a variant thereof having 1-5 amino acid modifications; the nucleic acid molecule of claim 1.

Also described herein is: a viral vector comprising a nucleic acid molecule described herein; a population of human T cells (e.g., a population comprising central memory T cells) transduced by a vector comprising a nucleic acid molecule described herein.

Also described herein is a method of treating can tumor or cancer that expresses a chlorotoxin receptor and/or an IL-13 receptor (e.g., IL-13Rα2) (including, e.g., glioblastoma, primary brain tumors and gliomas (glioblastoma multiforme WHO Grade IV, anaplastic astrocytoma WHO Grade III, low-grade astrocytoma WHO Grade II, pilocytic astrocytoma WHO Grade I, other ungraded gliomas, oligodendroglioma, gliosarcoma, ganglioglioma, meningioma, ependymona), neuroectodermal tumors (medulloblastoma, neuroblastoma, ganglioneuroma, melanoma (metastatic), melanoma (primary), pheochromocytoma, Ewing's sarcoma, primitive neuroectodermal tumors, small cell lung carcinoma, Schwannoma), other brain tumors (epidermoid cysts, brain tumors of unknown pathology, pituitary gland of glioblastoma multiforme pt., metastatic tumors to brain of unknown tissue origin), melanoma, melanoma metastases, breast cancer, breast cancer metastases, kidney cancer, kidney cancer metastases, liver cancer, liver cancer metastases, lung cancer, lung cancer metastases, lymphoma, lymphoma metastases, ovarian cancer, ovarian cancer metastases, pancreatic cancer, pancreatic cancer metastases, prostate cancer, prostate cancer metastases, colorectal cancer, colorectal cancer metastases, combinations thereof, and the like, in a patient comprising administering a population of autologous or allogeneic human T cells transduced by a vector comprising a nucleic acid molecule described herein. In various embodiments: the chimeric antigen receptor is administered locally or systemically; in some embodiments, a method of treatment includes cells expressing one or more of chlorotoxin receptor and/or IL-13Rα2, and the cells are cancerous cells; and the chimeric antigen receptor is administered by single or repeat dosing.

In various embodiments, the chimeric antigen receptor comprises: a huIL-13 (e.g., an IL-13 comprising the amino acid sequence GPVPPSTALRELIEELVNITQNQKAPLCNGSMVWSINLTAGMYCAALESLINVSGCSA IEKTQRMLSGFCPHKVSAGQFSSLHVRDTKIEVAQFVKDLLLHLKKLFREGRFN (SEQ ID NO:1) with up to 10 (e.g., 1, 2, 3, 4, 5, 6, 7, 8, 9 or 10) single amino acid substitutions). In some embodiments, the CAR comprises: a variant of a human IL13 having 1-10 amino acid modification that increase binding specificity for IL13Rα2 versus IL13Rα1 (e.g., IL13 E13Y (E13Y mutation shown bold and underlined):

(SEQ ID NO: 29)
GPVPPSTALRYLIEELVNITQNQKAPLCNGSMVWSINLTAGMY
CAALESLINVSGCSAIEKTQRMLSGFCPHKVSAGQFSSLHVRD
TKIEVAQFVKDLLLHLKKLFREGRFN.

In various embodiments, the chimeric antigen receptor comprises: a chlorotoxin (e.g., chlorotoxin comprising the amino acid sequence MCMPCFTTDHQMAKRCDDCCGGKGRGKCYGPQCLCR; SEQ ID NO:34) or a variant thereof having 1-5 (e.g., 1 or 2) amino acid modifications (e.g., substitutions) provided that the cysteine residues are not modified. In some embodiments, the CAR includes a toxin related to chlorotoxin instead of chlorotoxin. Thus, the CAR can include GaTx2, a toxin from Leiurus quinquestriatus hebraeus (VSCEDCPDHCSTQKARAKCDNDKCVCEPI; SEQ ID NO:35) or a variant thereof having 1-5 (e.g., 1 or 2) amino acid modifications (e.g., substitutions) provided that the cysteine residues are not modified; the CAR can include GaTx1, a toxin from Leiurus quinquestriatus hebraeus (CGPCFTTDHQMEQKCAECCGGIGKCYGPQCLCNR; SEQ ID NO:36) or a variant thereof having 1-5 (e.g., 1 or 2) amino acid modifications (e.g., substitutions) provided that the cysteine residues are not modified; the CAR can include AaCtx, a toxin from Androctonus australis (MCIPCFTTNPNMAAKCNACCGSRRGSCRGPQCIC; SEQ ID NO:37) or a variant thereof having 1-5 (e.g., 1 or 2) amino acid modifications (e.g., substitutions) provided that the cysteine residues are not modified; the CAR can include BmKCT, a toxin from Buthus martensii (CGPCFTTDANMARKCRECCGGIGKCFGPQCLCNRI; SEQ ID NO:38) or a variant thereof having 1-5 (e.g., 1 or 2) amino acid modifications (e.g., substitutions) provided that the cysteine residues are not modified. An additional embodiment the CAR comprises: a variant of a chlorotoxin having 1-5 amino acid modifications that increase binding specificity or immunogenicity for the chlorotoxin receptor (Cltx-R).

Also described are T cells harboring a vector expressing the CAR. In various embodiments: at least 20%, 30%, or 40% of the transduced human T cells are central memory T cells; at least 30% of the transduced human T cells are CD4+ and CD62L+ or CD8+ and CD62L+. In various embodiments: the population of human T cells comprise a vector expressing a chimeric antigen receptor comprising an amino acid sequence selected from SEQ ID NOs: 43-52 or a variant thereof having 1-5 amino acid modifications (e.g., 1 or 2) amino acid modifications (e.g., substitutions); the population of human T cells comprises central memory T cells (T_{CM} cells) e.g., at least 20%, 30%, 40%, 50% 60%, 70%, 80% of the cells are T_{CM} cells, or the population of T cells comprises a combination of central memory T cells, naïve T cells and stem central memory cells (T_{CM/SCM/N} cells) e.g., at least 20%, 30%, 40%, 50% 60%, 70%, 80% of the cells are T_{CM/SCM/N} cells. In some embodiments, the population of T cells includes both CD4+ cells and CD8+ cells (e.g., at least 20% of the CD3+ T cells are CD4+ and at least 3% of the CD3+ T cells are CD8+ and at least 70, 80 or 90% are either CD4+ or CD8+; at least 15%, 20%, 25%, 30%, 35%, 40%, 50%, 60% of the cells CD3+ cells are CD4+ and at least 4%, 5%, 8%, 10%, 20 of the CD3+ cells are CD8+ cells). In some embodiments, the population of human T cells are autologous to the patient. In some embodiments, the population of human T cells are allogenic to the patient.

Described herein is a nucleic acid molecule comprising a nucleotide sequence encoding a chimeric antigen receptor (CAR), wherein the chimeric antigen receptor comprises: a chlorotoxin domain, an IL-13 domain, a spacer, a transmembrane domain, a co-stimulatory domain, and a CD3ζ signaling domain, wherein the chlorotoxin domain can precede or follow the IL13 domain and wherein a linker is located between the chlorotoxin domain and the IL-13 domain.

In various embodiments transmembrane domain is comprises an amino acid sequence selected from the group consisting of SEQ ID NOs: 13-20; the IL-13 domain comprises the amino acid sequence of SEQ ID NO: 1, or variant thereof having 1-5 single amino acid modifications, and the amino acid sequence of SEQ ID NO: 34, or variant thereof having 1-5 single amino acid modifications; the co-stimulatory domain comprises an amino acid sequence selected from the group consisting of SEQ ID NOs: 22-25; the chlorotoxin domain, the IL-13 domain and the linker together comprises an amino acid selected from SEQ ID NOs: 39-42; a linker of 3 to 50 amino acids is located between the chlorotoxin domain and the IL-13 domain; the linker comprises an amino acid sequence selected from GGG and SEQ ID NOs: 30-33; a linker is located between the co-stimulatory domain and the CD3ζ signaling domain; the linker located between the co-stimulatory domain and the CD3ζ signaling domain consists of or comprises 3 to 10 amino acids; the co-stimulatory domain is 41-BB, CD28 or CD228gg and the spacer comprises an amino acid sequence selected from SEQ ID NOs: 2-12; the CAR comprises an IL-13 domain followed by a flexible linker followed by a chlorotoxin domain or a chlorotoxin domain followed by a flexible linker followed by an IL-13 domain; the chlorotoxin domain comprises SEQ ID NO:34; the IL-13 domain comprises SEQ ID NO:29; the CAR comprises an IL-13 domain followed by a flexible linker followed by a chlorotoxin domain; the CAR comprises an amino acid sequence selected from SEQ ID NOs: 39-42; the CAR comprises an amino acid sequence selected from SEQ ID NOs: 43-52; the CAR comprises the amino acid sequence of at least 90%, 95%, 96%, 97%, 98%, 99%, or 100% identical to any of SEQ ID NOs: 43-52; the CAR comprises the amino acid sequence of any one of SEQ ID NO: 43-52, or a variant thereof having 1-5 amino acid modifications; the spacer comprises the amino acid sequence of any one of SEQ ID NOs: 2-12, the transmembrane domains comprises the amino acid sequence of any one of SEQ ID NOs: 13-20, the co-stimulatory domain comprises the amino acid sequence of any one of SEQ ID NOs: 22-25; the CAR comprises the amino acid sequence of any one of SEQ NOs: 43-52, the amino acid sequence of any one of SEQ ID NOs: 10 and 11, the amino acid sequence of any one of SEQ ID NOs: 14, 16 and 17, the amino acid sequence of any one of SEQ ID NOs: 22-24 and the amino acid sequence of SEQ ID NO:21; and the linker between the IL-13 domain and the chlorotoxin domain comprises an amino acid sequence selected from SEQ ID NOs: 30-33.

Also described is a nucleic acid molecule comprising a nucleotide sequence encoding a chimeric antigen receptor (CAR), wherein the chimeric antigen receptor comprises: a toxin domain comprising an amino acid sequence selected from SEQ ID NOs: 35-38, an IL-13 domain, a spacer, a transmembrane domain, a co-stimulatory domain, and a CD3ζ signaling domain, wherein the chlorotoxin domain can precede or follow the IL13 domain and wherein a linker is located between the chlorotoxin domain and the IL-13 domain.

In various embodiments: the transmembrane domain is comprises an amino acid sequence selected from the group consisting of SEQ ID NOs: 13-20; the IL-13 domain comprises the amino acid sequence of SEQ ID NO: 33, or variant thereof having 1-5 single amino acid modifications, and the amino acid sequence of SEQ ID NO: 34, or variant thereof having 1-5 single amino acid modifications; the wherein the co-stimulatory domain comprises an amino acid sequence selected from the group consisting of SEQ ID NOs: 22-25; a linker of 3 to 50 amino acids is located between the toxin domain and the IL-13 domain; the linker comprises an amino acid sequence selected from GGG and SEQ ID NOs: 30-33; a linker is located between the co-stimulatory domain and the CD3ζ signaling domain; the linker located between the co-stimulatory domain and the CD3ζ signaling domain consists of or comprises 3 to 10 amino acids; the co-stimulatory domain is 41-BB, CD28 or CD228gg and the spacer comprises an amino acid sequence selected from SEQ ID NOs: 2-12; the CAR comprises an IL-13 domain followed by a flexible linker followed by a toxin domain or a toxin domain followed by a flexible linker followed by an IL-13 domain; the IL-13 domain comprises SEQ ID NO:29; the CAR comprises an IL-13 domain followed by a flexible linker followed by the toxin domain; the spacer comprises the amino acid sequence of any one of SEQ ID NOs: 2-12, the transmembrane domains comprises the amino acid sequence of any one of SEQ ID NOs: 13-20, the co-stimulatory domain comprises the amino acid sequence of any one of SEQ ID NOs: 22-25; the CAR comprises the amino acid sequence of any one of SEQ NOs: 43-52, the amino acid sequence of any one of SEQ ID NOs: 10 and 11, the amino acid sequence of any one of SEQ ID NOs: 14, 16 and 17, the amino acid sequence of any one of SEQ ID NOs: 22-24 and the amino acid sequence of SEQ ID NO:21; and the linker between the IL-13 domain and the toxin domain comprises an amino acid sequence selected from SEQ ID NOs: 30-33.

Also described is: an expression vector comprising a nucleic acid molecule described herein; a viral vector comprising a nucleic acid molecule described herein; a population of human T cells (e.g., comprising central memory T cells, naive memory T cells, and/or PBMC substantially depleted for CD25+ cells and CD14+ cells) or human NK cell harboring a nucleic acid molecule described herein.

Also described is a method of treating a tumor of neuroectodermal or a tumor of peripheral neuroectodermal tumor origin a glioma comprising administering a therapeutically effective amount of a population of autologous or allogeneic human T cells or NK cells harboring the nucleic acid molecule described herein. In various embodiments: the T cells or NK cells are administered locally or systemically; the T cells or NK cells are administered to intraventricularly; and the T cells or NK cells are administered by single or repeat dosing.

Also described is a method of preparing CART cells comprising: providing a population of autologous or allogeneic human T cells and transducing the T cells by a vector comprising the nucleic acid molecule described herein. In various embodiments: the T cell population is at least about 80%, 85%, 90%, 95%, 96%, 97%, 98%, 99%, or 100% CD14- and at least about 80%, 85%, 90%, 95%, 96%, 97%, 98%, 99%, or 100% CD25-; and the tumor is tumor is a glioblastoma.

### Bispecific CAR

A bispecific CAR described herein include a targeting domain comprising two subdomains. In some embodiments, a targeting domain comprising the amino acid sequence: MCMPCFTTDHQMARKCDDCCGGKGRGKCYGPQCLCRGGGGPVPPSTALRYLIEEL VNITQNQKAPLCNGSMVWSINLTAGMYCAALESLINVSGCSAIEKTQRMLSGFCPHK VSAGQFSSLHVRDTKIEVAQFVKDLLLHLKKLFREGRFN (SEQ ID NO:39) or comprising the sequence MCMPCFTTDHQMARKCDDCCGGKGRGKCYGPQCLCRGGGGSGGGGSGGGGSGPV PPSTALRYLIEELVNITQNQKAPLCNGSMVWSINLTAGMYCAALESLINVSGCSAIEK TQRMLSGFCPHKVSAGQFSSLHVRDTKIEVAQFVKDLLLHLKKLFREGRFN (SEQ ID NO:40) or comprising the sequence: GPVPPSTALRYLIEELVNITQNQKAPLCNGSMVWSINLTAGMYCAALESLINVSGCSA IEKTQRMLSGFCPHKVSAGQFSSLHVRDTKIEVAQFVKDLLLHLKKLFREGRFNGGG MCMPCFTTDHQMARKCDDCCGGKGRGKCYGPQCLCR (SEQ ID NO:41) or comprising the sequence: GPVPPSTALRYLIEELVNITQNQKAPLCNGSMVWSINLTAGMYCAALESLINVSGCSA IEKTQRMLSGFCPHKVSAGQFSSLHVRDTKIEVAQFVKDLLLHLKKLFREGRFNGGG GSGGGGSGGGGSMCMPCFTTDHQMARKCDDCCGGKGRGKCYGPQCLCR (SEQ ID NO:42) or comprising the sequence: GPVPPSTALRYLIEELVNITQNQKAPLCNGSMVWSINLTAGMYCAALESLINVSGCSA IEKTQRMLSGFCPHKVSAGQFSSLHVRDTKIEVAQFVKDLLLHLKKLFREGRFN (SEQ ID NO:29) and the sequence MCMPCFTTDHQMARKCDDCCGGKGRGKCYGPQCLCR (SEQ ID NO:34), in either order, joined by a flexible linker, e.g., a linker comprising 3-20 amino acids.

In some embodiments, a CAR comprises the amino acid sequence: GPVPPSTALRYLIEELVNITQNQKAPLCNGSMVWSINLTAGMYCAALESLINVSGCSA IEKTQRMLSGFCPHKVSAGQFSSLHVRDTKIEVAQFVKDLLLHLKKLFREGRFN (SEQ ID NO: 29) and the sequence MCMPCFTTDHQMARKCDDCCGGKGRGKCYGPQCLCR (SEQ ID NO:34) joined by a flexible linker such as GGGGSGGGGSGGGGS or GGG.

A useful bispecific CAR can consist of or comprises the amino acid sequence of SEQ ID NO: 48-52 (mature CAR lacking a signal sequence) or the bispecific CAR can consist of or comprise the amino acid sequence of SEQ ID NO: 43-47 (immature CAR having a GMCSFRa signal sequence). The CAR and can be expressed in a form that includes a signal sequence, e.g., a human GM-CSF receptor alpha signal sequence (MLLLVTSLLLCELPHPAFLLIP; SEQ ID NO:28). The CAR can be expressed with additional sequences that are useful for monitoring expression, for example, a T2A skip sequence and a truncated EGFRt or a T2A skip sequence followed by a truncated CD19t. Thus, the CAR can comprise or consist of the amino acid sequence of SEQ ID Nos: 43-52 or can comprise or consist of an amino acid sequence that is at least 95%, 96%, 97%, 98% or 99% identical to SEQ ID Nos: 43-52. The CAR can comprise or consist of the amino acid sequence of any of SEQ ID Nos 43-52 with up to 1, 2, 3, 4 or 5 amino acid changes (preferably conservative amino acid changes). The CAR can comprise SEQ ID NO: 29 with up to 1, 2, 3, 4 or 5 amino acid changes (preferably conservative amino acid changes) and SEQ ID NO:34 with up to 1, 2, 3, 4 or 5 amino acid changes (preferably conservative amino acid changes) joined by a flexible linker. The CAR can comprise SEQ ID NO:34 with up to 1, 2, 3, 4 or 5 amino acid changes (preferably conservative amino acid changes) and SEQ ID NO:29 with up to 1, 2, 3, 4 or 5 amino acid changes (preferably conservative amino acid changes) joined by a flexible linker.

In some embodiments, the nucleic acid encoding amino acid sequences SEQ ID NOs: 43-52 are codon optimized. In some embodiments, the nucleic acid encoding amino acid sequences SEQ ID NOs: 43-52are not codon optimized.

### Spacer Region

The CAR described herein can include a spacer located between the tumor-targeting domain (e.g., domain comprising both a chlorotoxin and a IL-13) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

The CAR described herein can include a spacer (also called linker) within the tumor-targeting domain (e.g., between a chloroxin and an IL-13). A variety of different spacers can be used. Some of them include g3 comprising three glycines, (g4s)n comprising n (n=1, 2, 3, 4, 5, 6, 7, 8, 9, or 10) number of repeats of five amino acids: GGGGS (SEQ ID NO: 30); (g4s)3: GGGGSGGGGSGGGGS (SEQ ID NO: 31); (g4s)4: GGGGSGGGGSGGGGSGGGGS (SEQ ID NO: 32); (g4s)5: GGGGSGGGGSGGGGSGGGGSGGGGS (SEQ ID NO: 33), or variants thereof. Table 1 below provides a number of additional spacers that can be used in the CARs described herein.

TABLE 1
Examples of Spacers

| Name | Length | Sequence |
|---|---|---|
| a3 | 3 aa | AAA |
| g3 | 3 aa | GGG |
| linker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge (S228P) + | 22 aa | ESKYGPPCPPCPGGGSSGGGSG (SEQ ID NO: 5) |
| linker | | |
| CD28 hinge | 39 aa | IEVMYPPPYLDNEKSNGTIIHVKGKHLCPSPLFPGPSKP |
| | | (SEQ ID NO: 6) |
| CD8 hinge-48aa | 48 aa | AKPTTTPAPRPPTPAPTIASQPLSLRPEACRPAAGGAVHT |
| | | RGLDFACD (SEQ ID NO: 7) |
| CD8 hinge-45aa | 45 aa | TTTPAPRPPTPAPTIASQPLSLRPEACRPAAGGAVHTRGL |
| | | DFACD (SEQ ID NO: 8) |
| IgG4(HL-CH3) | 129 aa | ESKYGPPCPPCPGGGSSGGGSGGQPREPQVYTLPPSQEEMTKNQVS |
| (includes S228P | | LTCLVKGFYPSDIAVEWESNGQPENNYKTTPPVLDSDGSFFLYSRL |
| in hinge) | | VTDKSRWQEGNVFSCSVMHEALHNHYTQKSLSLSLGK (SEQ ID |
| | | NO: 9) |
| IgG4 | 229 aa | ESKYGPPCPSCPAPEFEGGPSVFLFPPKPKDTLMISRTPEVTCVVV |
| (L235E, N297Q) | | DVSQEDPEVQFNWYVDGVEVHNAKTKPREEQFQSTYRVVSVLTVLHQ |
| | | DWLNGKEYKCKVSNKGLPSSIEKTISKAKGQPREPQVYTLPPSQEEM |
| | | TKNQVSLTCLVKGFYPSDIAVEWESNGQPENNYKTTPPVLDSDGSF |
| | | FLYSRLTVDKSRWQEGNVFSCSV MHEALHNHYTQKSLSLSLGK |
| | | (SEQ ID NO: 10) |
| IgG4(S228P, | 229 aa | ESKYGPPCPPCPAPEFEGGPSVFLFPPKPKDTLMISRTPEVTCVVV |
| L235E, N297Q) | | DVSQEDPEVQFNWYVDGVEVHNAKTKPREEQFQSTYRVVSVLTVLH |
| IgG4(PEQ) | | QDWLNGKEYKCKVSNKGLPSSIEKTISKAKGQPREPQVYTLPPSQEE |
| | | MTKNQVSLTCLVKGFYPSDIAVEWESNGQPENNYKTTPPVLDSDG |
| | | SFFLYSRLTVDKSRWQEGNVFSCSV MHEALHNHYTQKSLSLSLGK |
| | | (SEQ ID NO: 11) |
| IgG4(CH3) | 107 aa | GQPREPQVYTLPPSQEEMTKNQVSLTCLVKGFYPSDIAVEWESNG |
| | | QPENNYKTTPPVLDSDGSFFLYSRLTVDKSRWQEGNVFSCSVMHE |
| | | ALHNHYTQKSLSLSLGK (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise a IgG4 hinge region having the sequence ESKYGPPCPSCP (SEQ ID NO:4) or ESKYGPPCPPCP (SEQ ID NO:3). The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO:12). Thus, the entire linker/spacer region can comprise the sequence: ESKYGPPCPPCPGGGSSGGGSGGQPREPQVYTLPPSQEEMTKNQVSLTCLVKGFYPS DIAVEWESNGQPENNYKTTPPVLDSDGSFFLYSRLTVDKSRWQEGNVFSCSVMHEA LHNHYTQKSLSLSLGK (SEQ ID NO:11). In some cases, the spacer has 1, 2, 3, 4, or 5 single amino acid changes (e.g., conservative changes) compared to SEQ ID NO:11. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the. Table 2 includes examples of suitable transmembrane domains. Where a spacer region is present, the transmembrane domain (TM) is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 13) |
| CD28 | NM_006139 | 27 aa | FWVLVVVGGVLACYSLLVTVA |
| | | | FIIFWV |
| | | | (SEQ ID NO: 14) |
| CD28(M) | NM_006139 | 28 aa | MFWVLVVVGGVLACYSLLVTV |
| | | | AFIIFWV |
| | | | (SEQ ID NO: 15) |
| CD4 | M35160 | 22 aa | MALIVLGGVAGLLLFIGLGIF |
| | | | F |
| | | | (SEQ ID NO: 16) |
| CD8tm | NM_001768 | 21 aa | IYIWAPLAGTCGVLLLSLVIT |
| | | | (SEQ ID NO: 17) |
| CD8tm2 | NM_001768 | 23 aa | IYIWAPLAGTCGVLLLSLVIT |
| | | | LY |
| | | | (SEQ ID NO: 18) |
| CD8tm3 | NM_001768 | 24 aa | IYIWAPLAGTCGVLLLSLVIT |
| | | | LYC |
| | | | (SEQ ID NO: 19) |
| 41BB | NM_001561 | 27 aa | IISFFLALTSTALLFLLFF L |
| | | | TLRFSVV |
| | | | (SEQ ID NO: 20) |

### Costimulatory Domain

The costimulatory domain can be any domain that is suitable for use with a CD3 signaling domain. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 of 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD38 Domain and Examples of
Costimulatory Domains

| Name | Accession | Length | Sequence |
|---|---|---|---|
| CD33 | J04132.1 | 113 aa | RVKFSRSADAPAYQQ |
| | | | GQNQLYNELNLGRRE |
| | | | EYDVLDKRRGRDPEM |
| | | | GGKPRRKNPQEGLYN |
| | | | ELQKDKMAEAYSEIG |
| | | | MKGERRRGKGHDGLY |
| | | | QGLSTATKDTYDALH |
| | | | MQALPPR |
| | | | (SEQ ID NO: 21) |
| CD28 | NM_006139 | 42 aa | RSKRSRLLHSDYMNM |
| | | | TPRRPGPTRKHYQPY |
| | | | APPRDFAAYRS |
| | | | (SEQ ID NO: 22) |
| CD28gg* | NM_006139 | 42 aa | RSKRSRGGHSDYMNM |
| | | | TPRRPGPTRKHYQPY |
| | | | APPRDFAAYRS |
| | | | (SEQ ID NO: 23) |
| 41BB | NM_001561 | 42 aa | KRGRKKLLYIFKQPF |
| | | | MRPVQTTQEEDGCSC |
| | | | RFPEEEEGGCEL |
| | | | (SEQ ID NO: 24) |
| OX40 | | 42 aa | ALYLLRRDQRLPPDA |
| | | | HKPPGGGSFRTPIQE |
| | | | EQADAHSTLAKI |
| | | | (SEQ ID NO: 25) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and a short linker consisting of 2-10, e.g., 3 amino acids (e.g., GGG) is can be positioned between the costimulatory domain and the CD3ζ signaling domain.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO:21). In some cases, the CD3ζ signaling has 1, 2, 3, 4 of 5 amino acid changes (preferably conservative) compared to SEQ ID NO:21.

### Truncated EGFR

The CD3ζ signaling domain can be followed by a ribosomal skip sequence (e.g., LEGGGEGRGSLLTCGDVEENPGPR; SEQ ID NO:26) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVA FRGDSFTHTPPLDPQELDILKTVKEITGFLLIQAWPENRTDLHAFENLEIIRGRTKQHG QFSLAVVSLNITSLGLRSLKEISDGDVIISGNKNLCYANTINWKKLFGTSGQKTKIISN RGENSCKATGQVCHALCSPEGCWGPEPRDCVSCRNVSRGRECVDKCNLLEGEPREF VENSECIQCHPECLPQAMNITCTGRGPDNCIQCAHYIDGPHCVKTCPAGVMGENNTL VWKYADAGHVCHLCHPNCTYGCTGPGLEGCPTNGPKIPSIATGMVGALLLLLVVAL GIGLFM (SEQ ID NO:27). In some cases, the truncated EGFR has 1, 2, 3, 4 of 5 amino acid changes (preferably conservative) compared to SEQ ID NO:27.

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In some cases, the dual-targeting CAR can be produced using a vector in which the CAR open reading frame is followed by a T2A ribosome skip sequence and a truncated EGFR (EGFRt), which lacks the cytoplasmic signaling tail. In this arrangement, co-expression of EGFRt provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt incorporated in the dual-targeting CAR lentiviral vector can act as suicide gene to ablate the CAR+T cells in cases of treatment-related toxicity.

A CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

Various T cell subsets isolated from the patient can be transduced with a vector for CAR expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of a dual-targeting CAR as well as a non-immunogenic surface marker for in vivo detection, ablation, and potential ex vivo selection.

The activated/genetically modified bispecific central memory T cells can be expanded in vitro with IL-2/IL-15 and then cryopreserved. Additional methods of preparing CART cells can be found in PCT/US2016/043392.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety for any and all purposes. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1D depict representative schematics of CLTX/IL-13 CAR T-cells. FIG. 1A depicts a schematic of a representative lentiviral chimeric antigen receptor (CAR) cassette using CLTX and IL13 as the antigen-targeting domains. FIG. 1B shows a schematic depicting four representative dual-targeting CAR constructs differing in the orientations between CLTX and IL13 tumor-targeting domains, and linkers that separate CLTX from IL13, including g3 (3 amino acids) and (g4s)3 (15 amino acids). FIG. 1C depicts a representative schematic of a dual-targeting CAR, which comprises the extracellular chlorotoxin, IL-13, modifiable linker, and IgG4Fc (EQ) spacer domains, a transmembrane domain and, the cytoplasmic CD3ζ signaling domain and co-stimulatory domains. FIG. 1D shows results from experiments with representative bispecific CAR T cells where CD-19t and Fc are co-expression of the bispecific CAR and CD19t transgenes in transduced T cell subsets. Percentages of immunoreactive cells for transduced cells (“CAR”), compared with untransduced cells (“Mock”), its expression was stable through 14 day culture duration.

FIG. 2 depicts T cell activation using the degranulation marker CD107a, which indicates T cell activation, of T cells transduced with different dual-targeting CAR constructs activated against co-cultured GBM cells.

FIGS. 3A-3C shows results from killing/rechallenge experiments with representative dual-targeting T cells that demonstrated the ability of CLTX/IL-13 T cells to kill tumor cell lines (e.g., GBM). FIG. 3A depicts killing and rechallenge results using PBT003-4 cells. FIG. 3B depicts killing and rechallenge results using PBT030-2 cells. FIG. 3C depicts killing and rechallenge results using PBT106 cells. Plotted are the viable tumor cell numbers, which indicate the long-term killing potential of CAR T cells, and rechallenge occurred at 48 hour intervals (arrowheads).

FIG. 4 shows the potency of representative dual-targeting CAR T cells with different intracellular signaling domains. A schematic diagram depicts representative dual-targeting CAR construct differing in intracellular co-stimulatory domains, including CD28 and 41BB. Killing assay results of dual-targeting CAR T cells with different co-stimulatory domains against GBM tumor cells show the percentages of tumor cells killed by the T cells harboring different dual-targeting CAR constructs; killing percentages were calculated by comparing with tumor cells numbers co-cultured with the same amount of mock T cells.

FIGS. 5A-5C show representative in vivo antitumor effect of dual-targeting CAR T cells against orthotopic GBM xenograft. FIG. 5A depicts a schematic showing a representative orthotopic xenograft generation followed by T cell treatment in NSG mice. FIG. 5B depicts survival of mice bearing two independent GBM models receiving different treatments (tumor only; mock-transduced T cells; Tandem-28z or Tandem-BBz CAR T cells) plotted over a 256-day monitoring period.

FIGS. 6A-6E show the annotated amino acid sequences of representative dual-targeting CAR T cells: (A) Cltx-g3-IL13(E13Y)-IgG4(PEQ)-CD28TM-CD28gg-CD3z (SEQ ID NO: 43; SEQ ID NO: 44 without the MLLLVTSLLLCELPHPAFLLIP signal peptide sequence), (B) Cltx-(g4s)3-IL13(E13Y)-IgG4(PEQ)-CD28TM-CD28gg-CD3z (SEQ ID NO: 45; SEQ ID NO: 46 without the MLLLVTSLLLCELPHPAFLLIP signal peptide sequence), (C) IL13(E13Y)-g3-Cltx-IgG4(PEQ)-CD28TM-CD28gg-CD3z (SEQ ID NO: 47; SEQ ID NO: 48 without the MLLLVTSLLLCELPHPAFLLIP signal peptide sequence), (D) IL13(E13Y)-(G45)3-Cltx-IgG4(PEQ)-CD28TM-CD28gg-CD3z (SEQ ID NO: 49; SEQ ID NO: 50 without the MLLLVTSLLLCELPHPAFLLIP signal peptide sequence), (E) Cltx-(g4s)3-IL13(E13Y)-IgG4(PEQ)-CD4tm-41BB-CD3Z (E; SEQ ID NO: 51; SEQ ID NO: 52 without the MLLLVTSLLLCELPHPAFLLIP signal peptide sequence).

### DETAILED DESCRIPTION

In this disclosure the generation and anti-tumor efficacy of CAR comprising an IL-13 and a chlorotoxin are described. The bispecific CAR T cells exhibited potent cytotoxicity against multiple cancer lines. Regional intraperitoneal in vivo delivery of bispecific CAR T cells in GBM murine tumor models conferred elimination of antigen-positive disease and extension of overall survival.

The present disclosure also provides methods for treating subjects with a cancer or tumor expressing a chlorotoxin receptor and/or an IL-13Rα1 and/or IL-13Rα2 and/or IL-4R.

T cells expressing a CAR comprising chlorotoxin (or a variant thereof) and IL-13 (or a variant thereof) can be useful in treatment of cancers such as glioblastoma, as well as other cancers expressing a receptor for chlorotoxin (or a variant thereof) or a receptor for IL-13 (or a variant thereof), which include, but are not limited to: primary brain tumors and gliomas (glioblastoma multiforme WHO Grade IV, anaplastic astrocytoma WHO Grade III, low-grade astrocytoma WHO Grade II, pilocytic astrocytoma WHO Grade I, other ungraded gliomas, oligodendroglioma, gliosarcoma, ganglioglioma, meningioma, ependymona), neuroectodermal tumors (medulloblastoma, neuroblastoma, ganglioneuroma, melanoma (metastatic), melanoma (primary), pheochromocytoma, Ewing's sarcoma, primitive neuroectodermal tumors, small cell lung carcinoma, Schwannoma), other brain tumors (epidermoid cysts, brain tumors of unknown pathology, pituitary gland of glioblastoma multiforme pt., metastatic tumors to brain of unknown tissue origin), other tumors (breast cancer, breast cancer metastases, kidney cancer, kidney cancer metastases, liver cancer, liver cancer metastases, lung cancer, lung cancer metastases, lymphoma, lymphoma metastases, ovarian cancer, ovarian cancer metastases, pancreatic cancer, pancreatic cancer metastases, prostate cancer, prostate cancer metastases, colorectal cancer, colorectal cancer metastases, and combinations thereof), combinations thereof, and the like.

### EXAMPLES

The invention is further described in the following examples, which do not limit the scope of the invention described in the claims.

### Materials and Methods

The following materials and methods were used in the Examples set forth herein. Dual targeting CART cells of this disclosure are generally referred to as CLTX/IL-13 CAR and IL-13/CLTX CAR interchangeably throughout the disclosure and does not specifically indicate the orientation of the two domains.

### Cell Lines

The GBM cell lines (e.g., PBT-106, PBT-030-2, and PBT-003-4) were cultured in RPMI-1640 (Lonza) containing 20% fetal bovine serum (FBS, Hyclone) and 1× antibiotic-antimycotic (1× AA, Gibco) (complete RPMI). The cancer cell lines were cultured in Dulbecco's Modified Eagles Medium (DMEM, Life Technologies) containing 10% FBS, 1× AA, 25 mM HEPES (Irvine Scientific), and 2 mM L-Glutamine (Fisher Scientific) (complete DMEM). All cells were cultured at 37° C. with 5% CO2.

### DNA Constructs and Lentivirus Production

Tumor cells were engineered to express enhanced green fluorescent protein and firefly luciferase (eGFP/ffluc) by transduction with epHIV7 lentivirus carrying the eGFP/ffluc fusion under the control of the EF1α promoter as described previously (Brown et al, Cancer Res, 2009).

Lentivirus was generated as previously described (Brown et al, Mol Ther, 2018). Briefly, 293T cells were transfected with packaging plasmid and CAR lentiviral backbone plasmid using a modified calcium phosphate method. Viral supernatants were collected after 3 to 4 days and treated with 2 mM magnesium and 25 U/mL Benzonase® endonuclease (EMD Millipore). Supernatants were concentrated via high-speed centrifugation and lentiviral pellets were resuspended in phosphate-buffered saline (PBS)-lactose solution (4 g lactose per 100 mL PBS), aliquoted and stored at −80° C. Lentiviral titers were quantified using HT1080 cells based on CD19t expression or EGFRt expression.

### T Cell Isolation, Lentiviral Transduction, and Ex Vivo Expansion

Leukapheresis products were obtained from consented research participants (healthy donors) under protocols approved by the City of Hope Internal Review Board (IRB). On the day of leukapheresis, peripheral blood mononuclear cells (PBMC) were isolated by density gradient centrifugation over Ficoll-Paque (GE Healthcare) followed by multiple washes in PBS/EDTA (Miltenyi Biotec). Cells were rested overnight at room temperature (RT) on a rotator, and subsequently washed and resuspended in X-VIVO T cell medium (Lonza) containing 10% FBS (complete X-VIVO). Up to 5.0×10⁹ PBMC were incubated with anti-CD14 and anti-CD25 microbeads (Miltenyi Biotec) for 30 min at RT and magnetically depleted using the CliniMACS® system (Miltenyi Biotec) according to the manufacturer's protocol and these were termed depleted PBMCs (dPBMC). dPBMC were frozen in CryoStor® CS5 (StemCell Technologies) until further processing.

T cell activation and transduction was performed as described previously (Wang et al, Sci Transl Med, 2020). Briefly, freshly thawed dPBMC were washed once and cultured in complete X-VIVO containing 100 U/mL recombinant human IL-2 (rhIL-2, Novartis Oncology) and 0.5 ng/mL recombinant human IL-15 (rhIL-15, CellGenix). For CAR lentiviral transduction, T cells were cultured with CD3/CD28 Dynabeads® (Life Technologies), protamine sulfate (APP Pharmaceuticals), cytokine mixture (as stated above) and desired lentivirus at a multiplicity or infection (MOI) of 1 the day following bead stimulation. Cells were then cultured in and replenished with fresh complete X-VIVO containing cytokines every 2-3 days. After 7 days, beads were magnetically removed, and cells were further expanded in complete X-VIVO containing cytokines to achieve desired cell yield. CART cells were positively selected for CD19t or EGFRt using the EasySep™ CD19 Positive Enrichment Kit I or II (StemCell Technologies) according to the manufacturer's protocol. Following further expansion, cells were frozen in CryoStor® CS5 prior to in vitro functional assays and in vivo tumor models. Purity and phenotype of CAR T cells were verified by flow cytometry.

### Flow Cytometry

For flow cytometric analysis, cells were resuspended in FACS buffer (Hank's balanced salt solution without Ca2+, Mg2+, or phenol red (HBSS−/−, Life Technologies) containing 2% FBS and 1× AA). Cells were incubated with primary antibodies for 30 minutes at 4° C. in the dark. For secondary staining, cells were washed twice prior to 30 min incubation at 4° C. in the dark with either Brilliant Violet 510 (BV510), fluorescein isothiocyanate (FITC), phycoerythrin (PE), peridinin chlorophyll protein complex (PerCP), PerCP-Cy5.5, PE-Cy7, allophycocyanin (APC), or APC-Cy7 (or APC-eFluor780)-conjugated antibodies. Antibodies against CD3 (BD Biosciences, Clone: SK7), CD4 (BD Biosciences, Clone: SK3), CD8 (BD Biosciences, Clone: SK1), CD14 (BD Biosciences, Clone: MΦP9), CD19 (BD Biosciences, Clone: SJ25C1), CD25 (BD Biosciences, Clone: 2A3), mouse CD45 (BioLegend, Clone: 30-F11), CD45 (BD Biosciences, Clone: 2D1), CD69 (BD Biosciences, Clone: L78), CD137 (BD Biosciences, Clone: 4B4-1), MUC1 (BioLegend, Clone 16A), MUC16 (Abcam, Clone X75 or EPSISR23), biotinylated Protein-L (GenScript USA) (25), Fc ( ), Donkey Anti-Rabbit Ig (Invitrogen), Goat Anti-Mouse Ig (BD Biosciences), and/or streptavidin (BD Biosciences) were used. Cell viability was determined using 4′, 6-diamidino-2-phenylindole (DAPI, Sigma). Flow cytometry was performed on a MACSQuant Analyzer 10 (Miltenyi Biotec), and the data was analyzed with FlowJo software (v10, TreeStar).

### In Vitro Tumor Killing and Rechallenge Assays

For tumor killing assays, CAR T cells and tumor targets were co-cultured at indicated effector:tumor (E:T) ratios in complete X-VIVO in the absence of exogenous cytokines in 96-well plates for 24 to 72 h and analyzed by flow cytometry as described above. Tumor killing by CAR T cells was calculated by comparing CD45-negative cell counts relative to that observed when targets were co-cultured with Mock (untransduced) T cells. In some embodiments, rechallenge assays, 24-168 hours after completion of the killing assay, CAR T cells and tumor targets were again co-cultured at indicated effector:tumor (E:T) ratios in complete X-VIVO in the absence of exogenous cytokines in 96-well plates for 24 to 72 h and analyzed by flow cytometry as described above. In some embodiments, multiple rechallenge assays follow a killing assay. In a representative initial rechallenge assay, 24-168 hours after completion of the killing assay, CART cells and tumor targets were again co-cultured at indicated effector:tumor (E:T) ratios in complete X-VIVO in the absence of exogenous cytokines in 96-well plates for 24 to 72 h and analyzed by flow cytometry as described above. Afterwards, one or more subsequent rechallenge assays were conducted 24-168 hours after completion of the initial rechallenge assay, CART cells and tumor targets were again co-cultured at indicated effector:tumor (E:T) ratios in complete X-VIVO in the absence of exogenous cytokines in 96-well plates for 24 to 72 h and analyzed by flow cytometry as described above.

### In Vivo Tumor Studies

All animal experiments were performed under protocols approved by the City of Hope Institutional Animal Care and Use Committee. For in vivo tumor studies, GBM cells (5.0×10⁶) were prepared in a final volume of 500 μl HBSS−/− and engrafted in 6 to 8 week old female NSG mice by injection. In some embodiments, engraftment comprises intraperitoneal (i.p.) injection, subcutantous (s.c.) injection, or intravenous (i.v.) injection. Tumor growth was monitored at least once a week via biophotonic imaging (Xenogen, LagoX) and flux signals were analyzed with Living Image software (Xenogen). For imaging, mice were i.p. injected with 150 μL D-luciferin potassium salt (Perkin Elmer) suspended in PBS at 4.29 mg/mouse. Once flux signals reached desired levels, day 8 for OV90 and day 14 for OVCAR3, T cells were prepared in 1× PBS, and mice were treated with 500 μL i.p. or 200 μL intravenous (i.v.) injection of 5.0×10⁶ Mock or Cltx/IL-13 CAR T cells. In the GBM tumor model, we tested the impact of repeat treatment with i.v. Cltx/IL-13 CAR T cells starting at day 4. In some embodiments, this was followed by treatments at additional indicated days post tumor engraftment. Humane endpoints were used in determining survival. Mice were euthanized upon signs of distress such as a distended belly due to ascites, labored or difficulty breathing, apparent weight loss, impaired mobility, or evidence of being moribund. At pre-determined time points or at moribund status, mice were euthanized and tissues and/or ascites fluid were harvested and processed for flow cytometry and/or immunohistochemistry as described below.

Peripheral blood was collected from isoflurane-anesthetized mice by retro-orbital (RO) bleed through heparinized capillary tubes (Chase Scientific) into polystyrene tubes containing a heparin/PBS solution (1000 units/mL, Sagent Pharmaceuticals). Volume of each RO blood draw (approximately 120 μL/mouse) was recorded for cell quantification per 4 blood. Red blood cells (RBCs) were lysed with 1× Red Cell Lysis Buffer (Sigma) according to the manufacturer's protocol and then washed, stained, and analyzed by flow cytometry as described above. Cells from i.p. ascites fluid was collected from euthanized mice by injecting 5 mL cold 1× PBS into the i.p. cavity, which was drawn up via syringe and stored on ice until further processing. RBC-depleted ascites was washed, stained, and analyzed by flow cytometry for tumor-associated glycoprotein expression and CAR T cells using antibodies and methods described above.

### Example 1: Construction of Bispecific CLTX/IL-13 CAR T Cells Containing Differing Linkers and Differing in the Orientations Between CLTX and IL-13 Tumor-Targeting Domains

The studies described below show that dual-targeting CAR can be stably expressed on primary T cells.

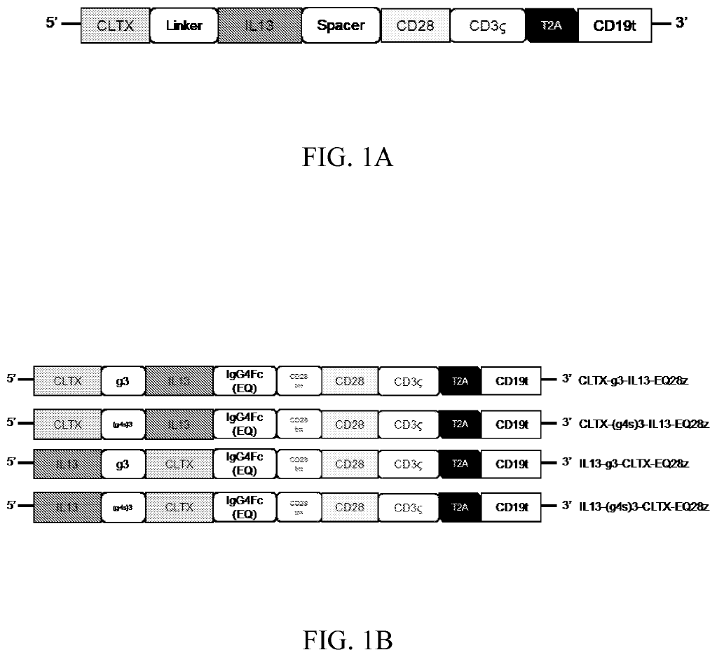

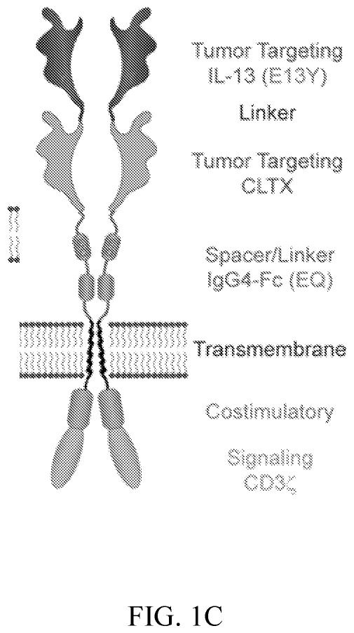

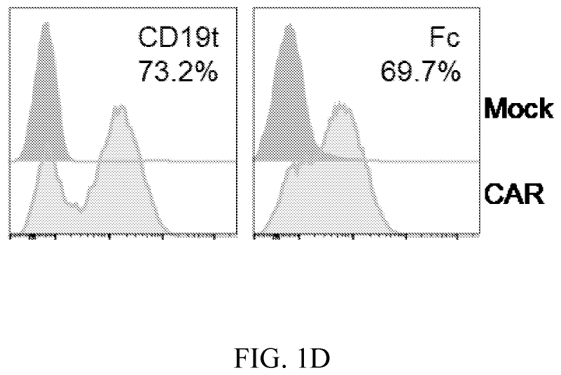

A number of bispecific CAR constructs were designed (FIGS. 1A-1C). A representative schematic of a lentiviral chimeric antigen receptor (CAR) cassette used depicts CLTX and IL-13 as the antigen-targeting domains, where transcription of the CLTX/IL-13 CAR, as well as the associated T2A ribosomal skip and truncated CD19 (CD19t) sequences were driven by the EF1 promoter (EF1p). The CAR constructs also included a transmembrane domain (TM), a costimulatory domain (e.g. CD28 or 41BB), a CD3 zeta domain. The CARs were co-expressed with truncated CD19t, which served as a marker for the successful transduction of the cells with the CAR construct.

Four representative CLTX/IL-13 CAR constructs differ in the orientations between CLTX and IL-13 tumor-targeting domains and/or the linker between these two domains (FIG. 1B). Without being bound by theory, differing lengths in the linker of the construct may provide differences in the CARs ability to bind an antigen and/or receptor and transmit activation signals after binding. Without being bound by theory, differing the orientation of the two tumor-targeting domains of the construct may provide differences in the CARs ability to bind an antigen and/or receptor and transmit activation signals after binding. These differences could also result differential killing of the targeted tumor cells.

In some embodiments, CLTX/IL-13 CAR lentivirus was used to transduce human healthy donor-derived peripheral blood mononuclear cells depleted of CD14+ and CD25+ cells (dPBMC) and/or T_{CM/SCM/N} cells, as previously described (Priceman S J, Gerdts E A, Tilakawardane D, Kennewick K T, Murad J P, Park A K, Jeang B, Yamaguchi Y, Yang X, Urak R, Weng L, Chang W C, Wright S, Pal S, Reiter R E, Wu A M, Brown C E, Forman S J.

Flow cytometric analysis of healthy donor T cells (HD417.1 T_{CM/SCM/N}) engineered to express the dual-targeting CAR show successful CAR expression (FIG. 1D). Anti-CD19 and anti-Fc staining showed sucessful co-expression of a representative CLTX/IL-13 CAR and CD19t transgenes in transduced T cell subsets. Percentages of immunoreactive cells for transduced cells (CLTX/IL-13 CAR), compared with untransduced cells (Mock), 14 days after CD3/CD28 bead stimulation are shown to prove the capability to transduce human T cells with a dual-targeting CAR (FIG. 1D).

### Example 2: Dual-Targeting CAR T Cells Activated Against Tumor Cells

The studies described below examined activation of dual-targeting CAR T cells with different targeting domain orientations and linker designs.

Schematic diagrams of representative dual-targeting CAR constructs differing in the orientations between CLTX and IL13 tumor-targeting domains, and linkers that separate CLTX with IL13, including g3 (3 amino acids) and (g4s)3 (15 amino acids) are shown in FIG. 1B.

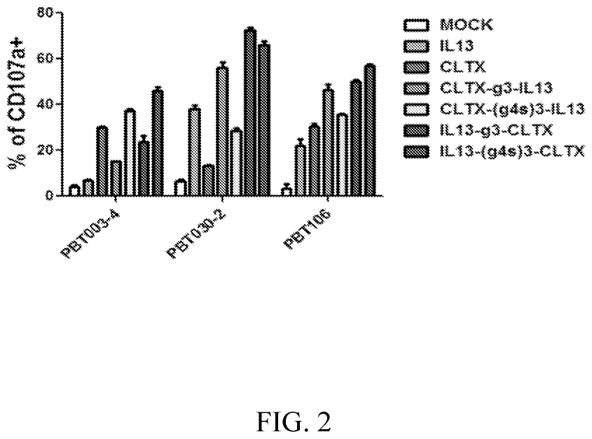

T cells transduced with different dual-targeting CAR constructs were activated against co-cultured GBM cells (FIG. 2). CAR T cells were cocultured with indicated GBM cells (PBT003-4, PBT030-2, and PBT106), at an effector:target (E:T) ratio=1:1 (20,000 T cells, 20,000 target cells) for 5 hours. Results show the percentage of CAR T cells that express the degranulation marker CD107a, which indicates T cell activation (FIG. 2). Results for untransduced (mock) T cells and T cells transduced with only CLTX or IL-13 are shown for comparison. Bispecific CAR T-cells were activated against all three GBM cell lines.

### Example 3: Validation that Dual-Targeting CAR T Cells Kill Tumor Cells

The studies described below examined effector potency of dual-targeting CAR T cells with different tumor-targeting domain orientations, linker designs, and costimulatory regions.

Schematic diagrams of representative dual-targeting CAR constructs differing in the orientations between CLTX and IL13 tumor-targeting domains, and linkers that separate CLTX with IL13, including g3 (3 amino acids) and (g4s)3 (15 amino acids) are shown in FIG. 1B

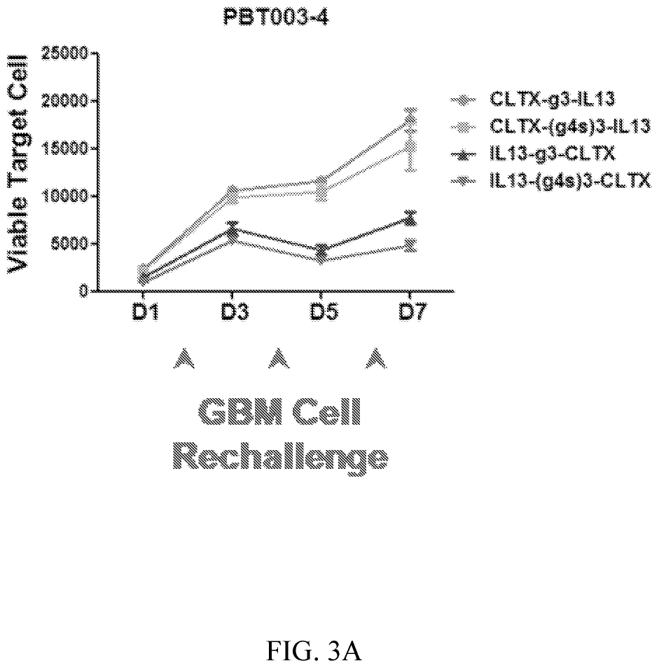

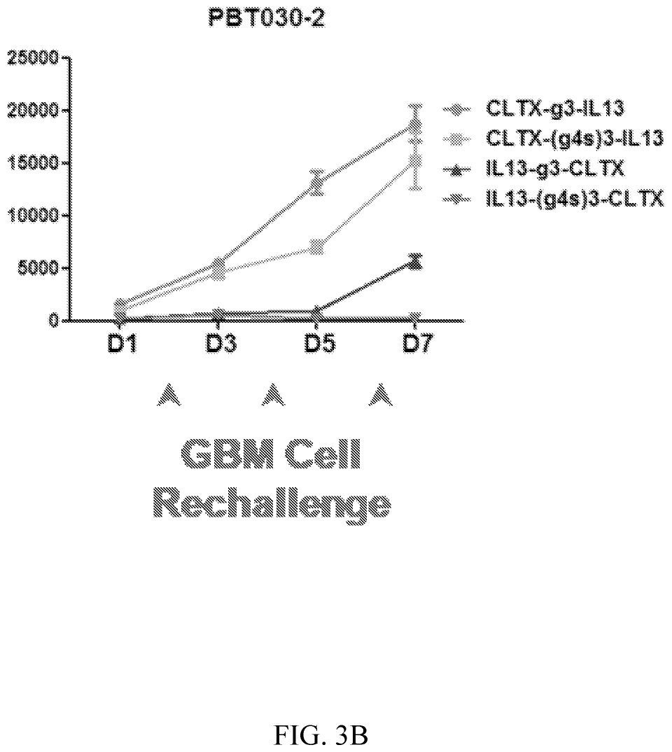

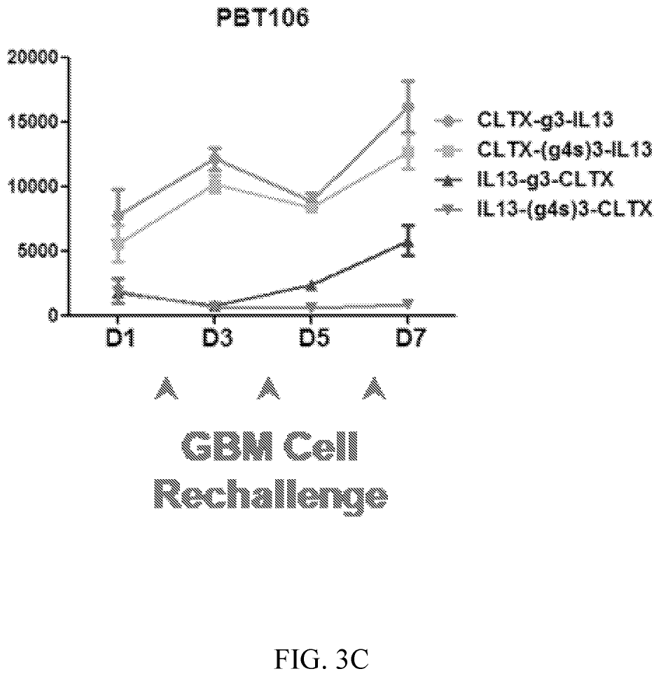

These dual-targeting T cells exhibit differential long-term effector function across different constructs (FIGS. 3A-3C). T cells engineered with different dual-targeted CAR constructs were co-cultured with GBM cells at an effector:target ratio=1:4 (4,000 T cells, 16,000 target cells), and rechallenged with 32,000 GBM cells every 48 hours (arrowheads). Plotted are the remaining viable tumor cell numbers, which indicate the long-term killing potential of the bispecific CAR T cells (FIGS. 3A-3C).

After 24 hours, T cell-mediated killing activity was evident with all four of the bispecific CAR T cells in the GBM tumor cell lines (PBT003-4 in FIG. 3A, PBT030-2 in FIG. 3B, and PBT106 in FIG. 3C) demonstrating the potent killing ability of these dual-targeting T CAR constructs. These dual-targeting CAR T cells also demonstrated potent killing activity in each rechallenge assay.

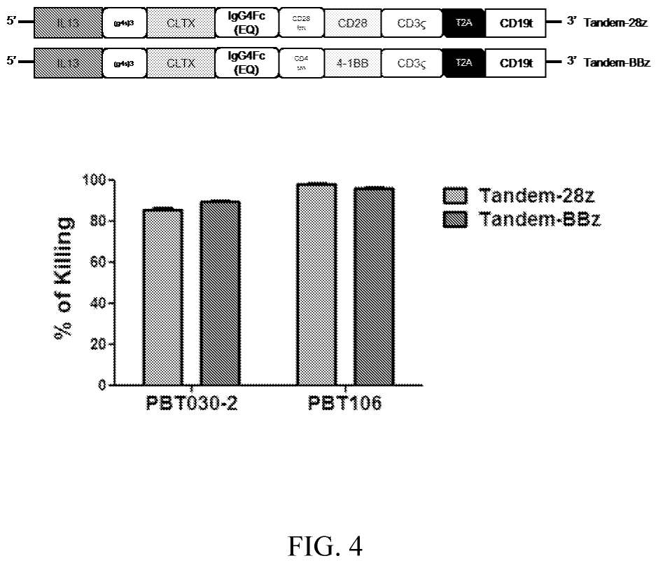

Experiments also tested effector potency of dual-targeting CAR T cells with different intracellular signaling domains. A schematic diagram of representative dual-targeting CAR constructs differing in intracellular co-stimulatory domains, including CD28 and 41BB, is shown in FIG. 4.

Using flow cytometry, we then tested for the killing of tumor cells (% specific lysis) of GBM tumor cells (including PBT030-2 and PBT106 cells) after 48 hours. Tumor cells were co-cultured with the indicated bispecific CAR T cells at an effector:target ratio=1:4 (4,000 T cells, 16,000 target cells) for 48 hours, and killing percentages were calculated by comparing with tumor cells numbers co-cultured with the same amount of mock T cells. The results show percentages of tumor cells killed by the dual-targeting CAR T cells. Both the CAR T cells with the CD28 domain and the CAR T cells with 41BB domain effectively killed GBM tumor cells (including PBT030-2 and PBT106 cells) (FIG. 4). Dual-targeting CAR T cells with different co-stimulatory domains are able to eliminate GBM cells.

All bispecific CAR T cells used herein led to effective, potent, and/or sustained killing of one or more of the GBM tumor cells lines.

### Example 4: Validation that Bispecific CLTX/IL-13 CAR T Cells Delivered In Vivo in a Mouse Model Exhibit Potent Anti-Tumor Activity and Confer Extended Lifespan to the Mice

To evaluate therapeutic potential and in vivo efficacy of representative dual-targeting CAR T cells to selectively target and eliminate tumor cells an in vivo model, CLTX/IL-13 CAR T cells were delivered to a huGBM mouse model, and tumor size and survival was evaluated over time.

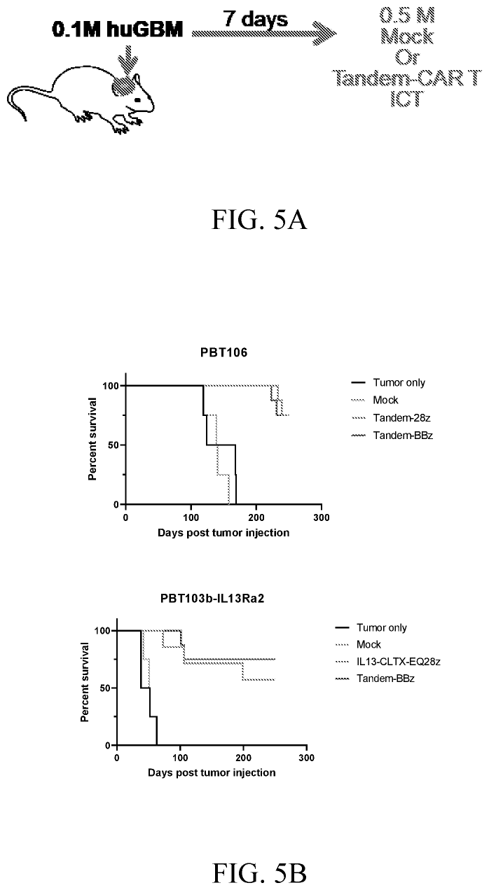

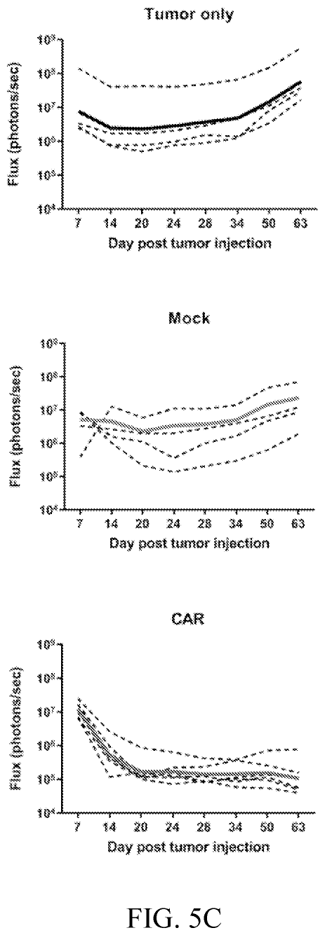

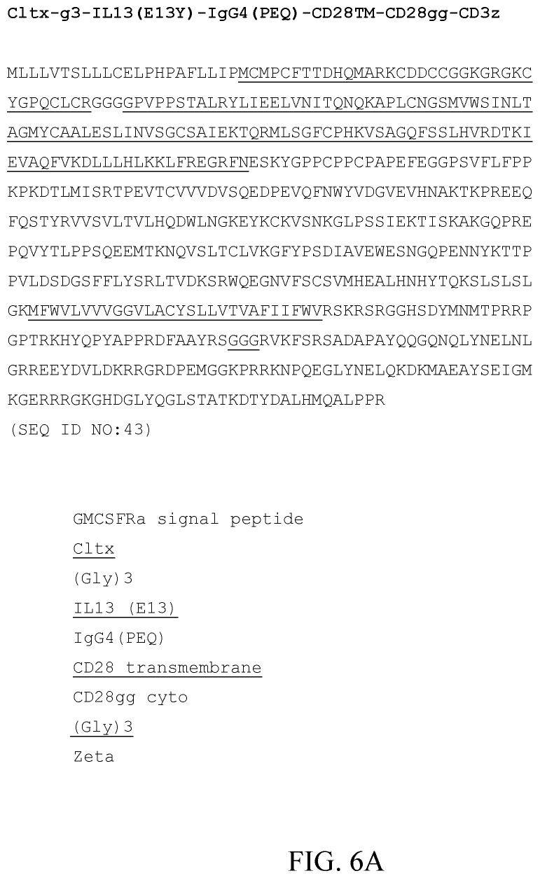

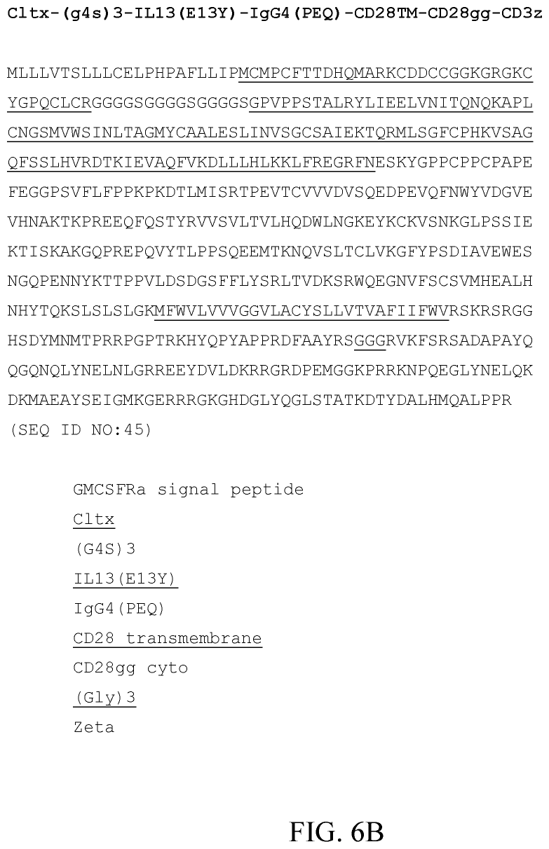

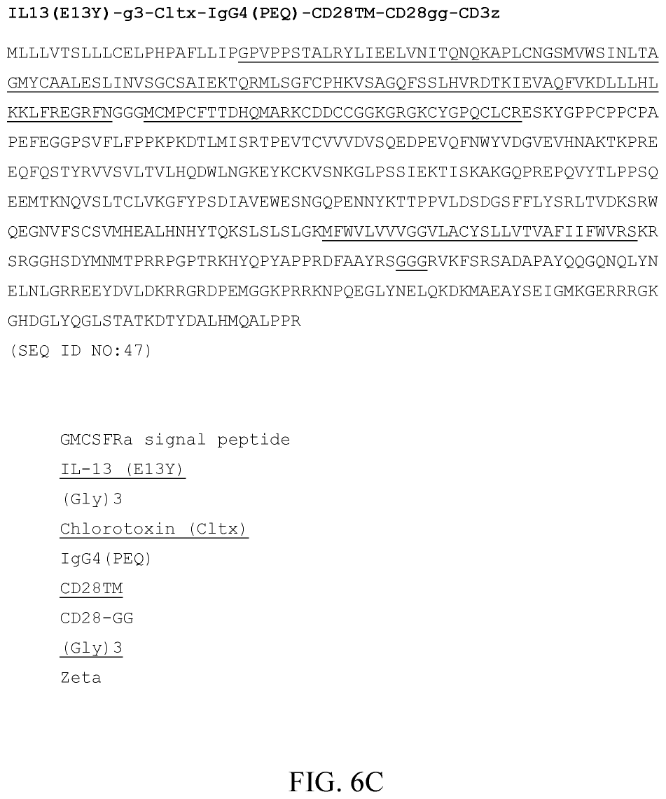

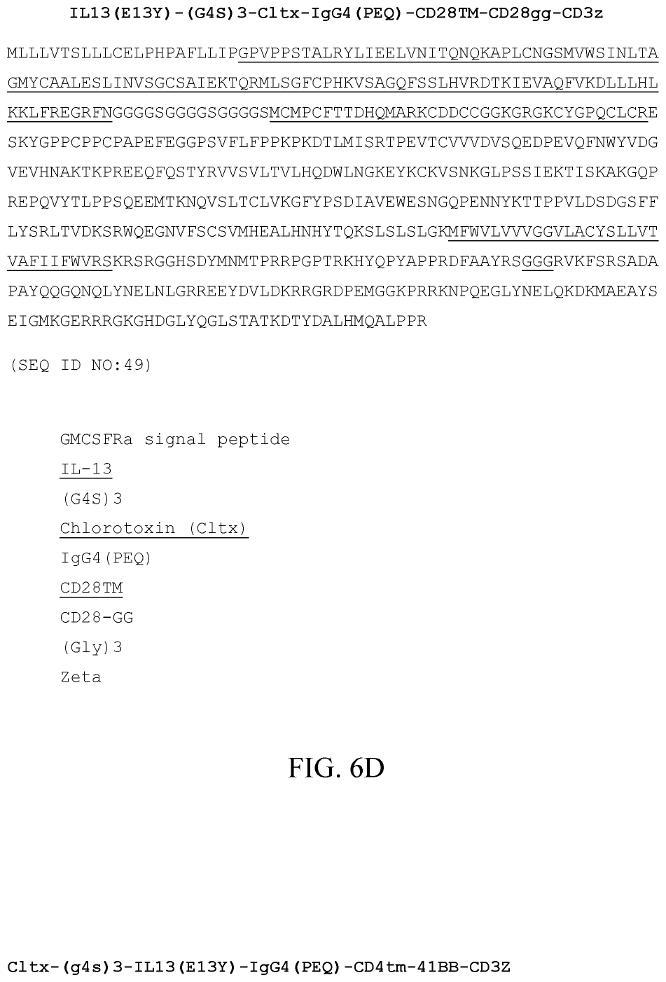

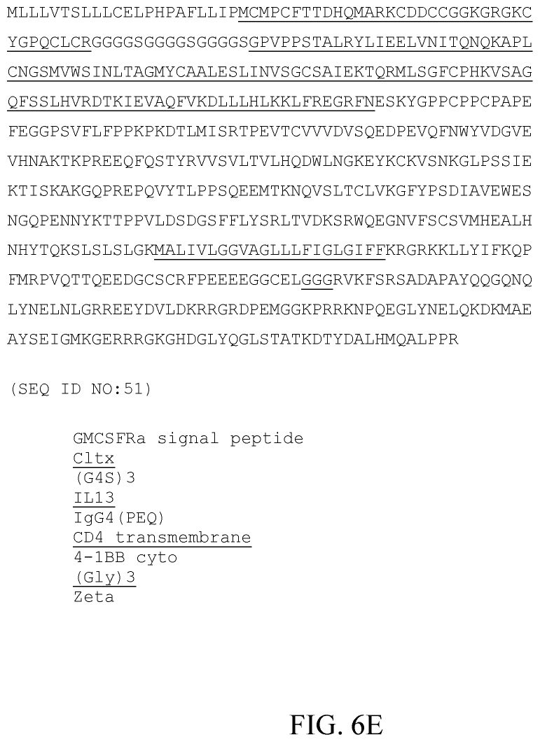

Experiments were conducted to show the antitumor effect of dual-targeting CAR T cells against orthotopic GBM xenograft. A schematic shows a representative model used herein: PBT106 orthotopic xenograft generation and CAR T cell treatment in NSG mice (FIG. 5A). Intracranial engraftment of PBT106 or PBT103B-IL13Ra2 GBM cells (100,000/mouse) were allowed to grow for 7 days before treating with Mock T cells or dual-targeting CAR T cells (500,000/mouse). Humane endpoints were used in determining survival curves of NSG mice engrafted with GBM cells and treated with T cells.

Survival of GBM-bearing mice receiving different treatments was plotted over a 256-day monitoring period (FIG. 5B). Mice with tumor growth were euthanized within 24 hours after discovery of neurological symptoms. Results show that the mice treated with dual-targeting CAR T cells (either Tandem-28z or Tandem-BBz) had significantly improved survival of human GBM bearing mice. Delivery of a composition comprising bispecific CAR T cells significantly extended survival of mice (FIG. 5B).

Additionally, tumor growth in each group of mice was monitored through bioluminescent imaging (FIG. 5C). GBM cells were lentivirally transduced to express ffluc to allow for tracking of tumor growth via non-invasive optical imaging. The bioluminescent intensity of each mouse (dotted lines) and the geometric means (solid lines) within each group report on the tumor size associated with each group (untreated, “tumor only”; untransduced, “mock”; bispecific CAR T cell treated, “CAR”). Rapid anti-tumor effects were observed in mice treated with the dual-targeting CAR2 T cells, reaching a maximal anti-tumor response 1-3 weeks following treatment (FIG. 5C).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All references are herein incorporated in their entirety for any and all purposes.

1. A nucleic acid molecule comprising a nucleotide sequence encoding a chimeric antigen receptor (CAR), wherein the chimeric antigen receptor comprises: a chlorotoxin domain, an IL-13 domain, a spacer, a transmembrane domain, a co-stimulatory domain, and a CD3ζ signaling domain, wherein a linker is located between the chlorotoxin domain and the IL-13 domain.

2. The nucleic acid molecule of claim 1, wherein the transmembrane domain is comprises an amino acid sequence selected from the group consisting of SEQ ID NOs: 13-20.

3. The nucleic acid molecule of claim 1, wherein the IL-13 domain comprises the amino acid sequence of SEQ ID NO: 1, or variant thereof having 1-5 single amino acid substitutions, or the amino acid sequence of SEQ ID NO: 29, or variant thereof having 1-5 single amino acid substitutions.

4. The nucleic acid molecule of claim 1, wherein the wherein the co-stimulatory domain comprises an amino acid sequence selected from the group consisting of SEQ ID NOs: 22-25.

5. (canceled)

6. The nucleic acid molecule of claim 1, wherein a linker of 3 to 50 amino acids is located between the chlorotoxin domain and the IL-13 domain.

7.-10. (canceled)

11. The nucleic acid molecule of claim 1, wherein the IL-13 domain is followed by the linker that is followed by the chlorotoxin domain, or wherein the chlorotoxin domain is followed by the linker that is followed by the IL-13 domain.

12. The nucleic acid molecule of claim 1, wherein the chlorotoxin domain comprises SEQ ID NO:34.

13.-15. (canceled)

16. The nucleic acid molecule of claim 1, wherein the CAR comprises an amino acid sequence selected from SEQ ID NOs: 43-52.

17.-21. (canceled)

22. An expression vector comprising the nucleic acid molecule of claim 1.

23. (canceled)

24. A population of human T cells or human NK cell harboring the nucleic acid molecule of claim 1.

25. The population of human T cells of claim 24, wherein the population of human T cells comprise central memory T cells, naive memory T cells, and/or PBMC substantially depleted for CD25+ cells and CD14+ cells.

26. A method of treating a tumor of neuroectodermal or a tumor of peripheral neuroectodermal tumor origin a glioma comprising administering a therapeutically effective amount of a population of autologous or allogeneic human T cells or NK cells harboring the nucleic acid molecule of claim 1.

27.-29. (canceled)

30. A method of preparing CAR T cells comprising: providing a population of autologous or allogeneic human T cells and transducing the T cells by a vector comprising the nucleic acid molecule of claim 1.

31. (canceled)

32. The method of claim 26, wherein the tumor is tumor is a glioblastoma.

33. A nucleic acid molecule comprising a nucleotide sequence encoding a chimeric antigen receptor (CAR), wherein the chimeric antigen receptor comprises: a toxin domain comprising an amino acid sequence selected from SEQ ID NOs: 35-38, an IL-13 domain, a spacer, a transmembrane domain, a co-stimulatory domain, and a CD3ζ signaling domain, wherein the chlorotoxin domain can precede or follow the IL13 domain and wherein a linker is located between the chlorotoxin domain and the IL-13 domain.

34.-49. (canceled)

50. A population of human T cells or human NK cell harboring the nucleic acid molecule of claim 33.

51. (canceled)

52. A method of treating a tumor of neuroectodermal or a tumor of peripheral neuroectodermal tumor origin or a glioma comprising administering a therapeutically effective amount of a population of autologous or allogeneic human T cells or NK cells harboring the nucleic acid molecule of claim 33.

53.-55. (canceled)

56. A method of preparing CAR T cells comprising: providing a population of autologous or allogeneic human T cells and transducing the T cells by a vector comprising the nucleic acid molecule of claim 33.

57.-58. (canceled)

59. A chimeric antigen receptor (CAR) comprising: a chlorotoxin domain, an IL-13 domain, a spacer, a transmembrane domain, a co-stimulatory domain, and a CD3ζ signaling domain, wherein a linker is located between the chlorotoxin domain and the IL-13 domain.

60. A population of human T cells or human NK cell expressing the CAR of claim 59.


SEQUENCE LISTING (52 sequences)

SEQ ID NO: 1 - protein, 112 aa, Artificial Sequence
  IL-13 fragment
GPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 2 - protein, 10 aa, Artificial Sequence
  spacer
GGGSSGGGSG

SEQ ID NO: 3 - protein, 12 aa, Artificial Sequence
  spacer
ESKYGPPCPPCP

SEQ ID NO: 4 - protein, 12 aa, Artificial Sequence
  spacer
ESKYGPPCPSCP

SEQ ID NO: 5 - protein, 22 aa, Artificial Sequence
  spacer
ESKYGPPCPPCPGGGSSGGGSG

SEQ ID NO: 6 - protein, 39 aa, Artificial Sequence
  spacer
IEVMYPPPYLDNEKSNGTIIHVKGKHLCPSPLFPGPSKP

SEQ ID NO: 7 - protein, 48 aa, Artificial Sequence
  spacer
AKPTTTPAPRPPTPAPTIASQPLSLRPEACRPAAGGAVHTRGLDFACD

SEQ ID NO: 8 - protein, 45 aa, Artificial Sequence
  spacer
TTTPAPRPPTPAPTIASQPLSLRPEACRPAAGGAVHTRGLDFACD

SEQ ID NO: 9 - protein, 129 aa, Artificial Sequence
  spacer
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, Artificial Sequence
  spacer
ESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Artificial Sequence
  spacer
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Artificial Sequence
  spacer
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Artificial Sequence
  TM domain
LCYLLDGILFIYGVILTALFL

SEQ ID NO: 14 - protein, 27 aa, Artificial Sequence
  TM domain
FWVLVVVGGVLACYSLLVTVAFIIFWV

SEQ ID NO: 15 - protein, 28 aa, Artificial Sequence
  TM domain
MFWVLVVVGGVLACYSLLVTVAFIIFWV

SEQ ID NO: 16 - protein, 22 aa, Artificial Sequence
  TM domain
MALIVLGGVAGLLLFIGLGIFF

SEQ ID NO: 17 - protein, 21 aa, Artificial Sequence
  TM domain
IYIWAPLAGTCGVLLLSLVIT

SEQ ID NO: 18 - protein, 23 aa, Artificial Sequence
  TM domain
IYIWAPLAGTCGVLLLSLVITLY

SEQ ID NO: 19 - protein, 24 aa, Artificial Sequence
  TM domain
IYIWAPLAGTCGVLLLSLVITLYC

SEQ ID NO: 20 - protein, 27 aa, Artificial Sequence
  TM domain
IISFFLALTSTALLFLLFFLTLRFSVV

SEQ ID NO: 21 - protein, 112 aa, Artificial Sequence
  Intracellular domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Artificial Sequence
  Intracellular domain
RSKRSRLLHSDYMNMTPRRPGPTRKHYQPYAPPRDFAAYRS

SEQ ID NO: 23 - protein, 41 aa, Artificial Sequence
  Intracellular domain
RSKRSRGGHSDYMNMTPRRPGPTRKHYQPYAPPRDFAAYRS

SEQ ID NO: 24 - protein, 42 aa, Artificial Sequence
  Intracellular domain
KRGRKKLLYIFKQPFMRPVQTTQEEDGCSCRFPEEEEGGCEL

SEQ ID NO: 25 - protein, 42 aa, Artificial Sequence
  Intracellular domain
ALYLLRRDQRLPPDAHKPPGGGSFRTPIQEEQADAHSTLAKI

SEQ ID NO: 26 - protein, 24 aa, Artificial Sequence
  T2A skip
LEGGGEGRGSLLTCGDVEENPGPR

SEQ ID NO: 27 - protein, 354 aa, Artificial Sequence
  Truncated EGFR
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 28 - protein, 22 aa, Artificial Sequence
  Signal sequence
MLLLVTSLLLCELPHPAFLLIP

SEQ ID NO: 29 - protein, 112 aa, Artificial Sequence
  IL-13 variant
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 30 - protein, 5 aa, Artificial Sequence
  spacer
GGGGS

SEQ ID NO: 31 - protein, 15 aa, Artificial Sequence
  spacer
GGGGSGGGGSGGGGS

SEQ ID NO: 32 - protein, 20 aa, Artificial Sequence
  spacer
GGGGSGGGGSGGGGSGGGGS

SEQ ID NO: 33 - protein, 25 aa, Artificial Sequence
  spacer
GGGGSGGGGSGGGGSGGGGSGGGGS

SEQ ID NO: 34 - protein, 36 aa, Artificial Sequence
  toxin
MCMPCFTTDHQMARKCDDCCGGKGRGKCYGPQCLCR

SEQ ID NO: 35 - protein, 29 aa, Artificial Sequence
  toxin
VSCEDCPDHCSTQKARAKCDNDKCVCEPI

SEQ ID NO: 36 - protein, 34 aa, Artificial Sequence
  toxin
CGPCFTTDHQMEQKCAECCGGIGKCYGPQCLCNR

SEQ ID NO: 37 - protein, 34 aa, Artificial Sequence
  toxin
MCIPCFTTNPNMAAKCNACCGSRRGSCRGPQCIC

SEQ ID NO: 38 - protein, 35 aa, Artificial Sequence
  toxin
CGPCFTTDANMARKCRECCGGIGKCFGPQCLCNRI

SEQ ID NO: 39 - protein, 151 aa, Artificial Sequence
  Dual targeting domain
MCMPCFTTDHQMARKCDDCCGGKGRGKCYGPQCLCRGGGGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 40 - protein, 163 aa, Artificial Sequence
  Dual targeting domain
MCMPCFTTDHQMARKCDDCCGGKGRGKCYGPQCLCRGGGGSGGGGSGGGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 41 - protein, 151 aa, Artificial Sequence
  Dual targeting domain
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGMCMPCFTTDHQMARKCDDCCGGKGRGKCYGPQCLCR

SEQ ID NO: 42 - protein, 163 aa, Artificial Sequence
  Dual targeting domain
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSMCMPCFTTDHQMARKCDDCCGGKGRGKCYGPQCLCR

SEQ ID NO: 43 - protein, 586 aa, Artificial Sequence
  Chimeric antigen receptor
MLLLVTSLLLCELPHPAFLLIPMCMPCFTTDHQMARKCDDCCGGKGRGKCYGPQCLCRGGGGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 564 aa, Artificial Sequence
  Chimeric antigen receptor
MCMPCFTTDHQMARKCDDCCGGKGRGKCYGPQCLCRGGGGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 598 aa, Artificial Sequence
  Chimeric antigen receptor
MLLLVTSLLLCELPHPAFLLIPMCMPCFTTDHQMARKCDDCCGGKGRGKCYGPQCLCRGGGGSGGGGSGGGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 576 aa, Artificial Sequence
  Chimeric antigen receptor
MCMPCFTTDHQMARKCDDCCGGKGRGKCYGPQCLCRGGGGSGGGGSGGGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 586 aa, Artificial Sequence
  Chimeric antigen receptor
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGMCMPCFTTDHQMARKCDDCCGGKGRGKCYGPQCLCRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 564 aa, Artificial Sequence
  Chimeric antigen receptor
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGMCMPCFTTDHQMARKCDDCCGGKGRGKCYGPQCLCRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 598 aa, Artificial Sequence
  Chimeric antigen receptor
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSMCMPCFTTDHQMARKCDDCCGGKGRGKCYGPQCLCRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 576 aa, Artificial Sequence
  Chimeric antigen receptor
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSMCMPCFTTDHQMARKCDDCCGGKGRGKCYGPQCLCRESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 593 aa, Artificial Sequence
  Chimeric antigen receptor
MLLLVTSLLLCELPHPAFLLIPMCMPCFTTDHQMARKCDDCCGGKGRGKCYGPQCLCRGGGGSGGGGSGGGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 571 aa, Artificial Sequence
  Chimeric antigen receptor
MCMPCFTTDHQMARKCDDCCGGKGRGKCYGPQCLCRGGGGSGGGGSGGGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR